(19) United States
(12) Patent Application Publication
(10) Pub. No.: US 2010/0234632 A1
(43) Pub. Date: Sep. 16, 2010

(54) PROCESS FOR THE PREPARATION OF ENTACAPONE

(75) Inventors: Pandurang Balwant Deshpande, Gujarat (IN); Anand Kumar Randey, Gujarat (IN); Dharmesh Ramniklal Dhameliya, Gujarat (IN); Bhimsing Rathod Dayawant, Gujarat (IN); Parven Kumar Luthra, Gujarat (IN)

Correspondence Address: MERCHANT & GOULD PC, P.O. BOX 2903, MINNEAPOLIS, MN, 55402-0903, US

(73) Assignee: Alembic Limited

(21) Appl. No.: 12/377,643

(22) PCT Filed: Aug. 13, 2007
(86) PCT No.: PCT/IN07/00345

§ 371 Date: Feb. 16, 2009

(30) Foreign Application Priority Data
Aug. 18, 2006 (IN) 1300/MUM/2006

(51) Int. Cl. C07C 253/30

(52) U.S. Cl. 558/371

(57) ABSTRACT

The present invention relates to an improved process for the preparation of Entacapone of formula (I)

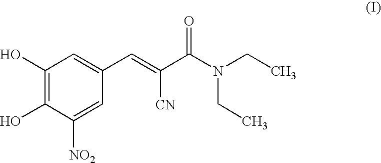

comprising a step of,
condensation of 3,4-dihydroxy-5-nitrobenzaldehyde of formula (II)

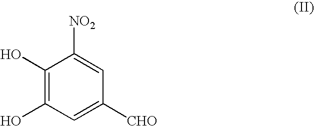

with N,N-diethylcyanoacetamide of formula (III)

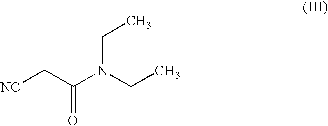

in the presence of two component solvent system, a catalyst and optionally a phase transfer catalyst to give Entacapone of formula (I).

### FIELD OF THE INVENTION

The present invention relates to an improved process for the preparation of Entacapone of formula (I)

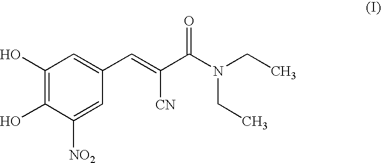

### BACKGROUND OF THE INVENTION

The chemical name of Entacapone is N,N-diethyl-2-cyano-3-(3,4-dihydroxy-5-nitrophenyl)acrylamide or (E)-2-cyano-3-(3,4-dihydroxy-5-nitrophenyl)-N,N-diethyl-2-propenamide and molecular formula is C₁₄H₁₅N₃O₅ and molecular weight is 305.29. Entacapone is marketed by Orion Corporation under tradename Comtan® and is indicated for the treatment of Parkinson's disease.

Entacapone is a potent and specific peripheral catechol-O-methyltransferase (COMT) inhibitor. It is used in combination with levodopa/carbidopa to treat Parkinson's disease, sometime referred to as shaking palsy. Entacapone enhances the effect of levedopa/carbidopa by improving muscle control.

U.S. Pat. No. 4,963,590 describes a process for the preparation of Entacapone of formula (I). The synthetic process disclosed in this patent comprises the condensation of 3,4-dihydroxy-5-nitrobenzaldehyde of formula (II) and N,N-diethylcyanoacetamide of formula (III) in anhydrous ethanol as shown below in Scheme-I

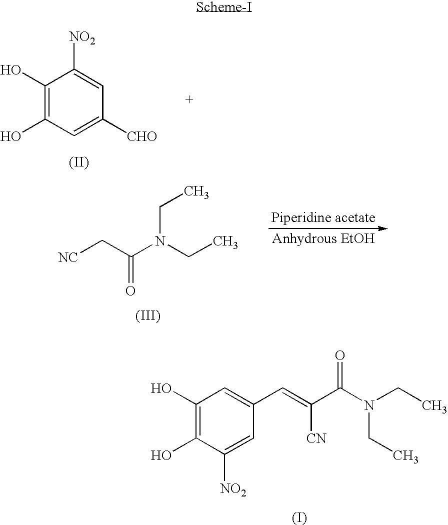

In the above process piperidine acetate was used as catalyst. Entacapone thus synthesized was obtained in 73% yield having a mixture of two geometrical isomeric forms, i.e., (E) and (Z). Moreover, the reaction is lengthy and takes long time which makes the process operation difficult.

Subsequently it is described in the U.S. Pat. No. 5,135,950 about preparing E-isomer and polymorphism-A from the mixture obtained from the reaction is reported in the GB patent No 2200109. It also discloses about the (E) and (Z)-isomers having the structural formula:

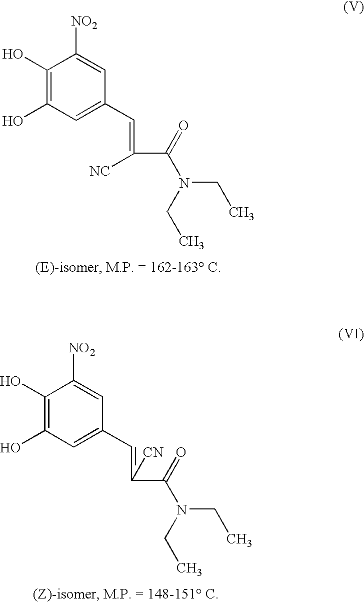

are obtained as mixture in the ratio of about 70-80% to about 30-20%, respectively.

U.S. Pat. No. 5,135,950 discloses that “crystallographically essentially pure” and stable polymorphic form A of (E)-N,N-diethyl-2-cyano-3-(3,4-dihydroxy-5-nitrophenyl)acrylamide is prepared by recrystallizing crude Entacapone from lower aliphatic carboxylic acids such as formic acid or acetic acid with a catalytic amount of hydrochloric or hydrobromic acid as shown below in Scheme-II

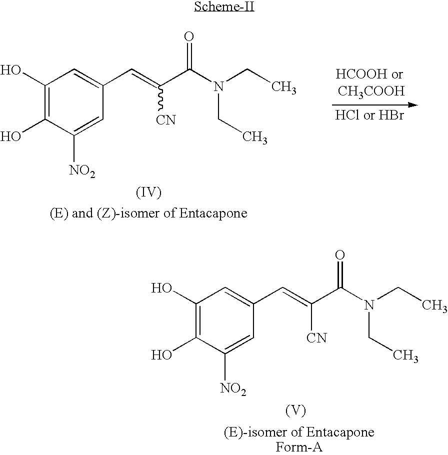

However, this process of isomerization using HBr/Acetic acid suffers with major drawback of operation difficulty as it requires specifically designed glass reactor because of the use of corrosive material. Moreover, it also involves high degree of temperature in highly acidic medium. Further, because of the low reaction volume, it is operationally difficult to transfer the final compound from the reactor.

In summary, process disclosed in prior art for the preparation of Entacapone, are tedious, time consuming and operationally difficult at industrial scale. Moreover, Entacapone obtained by prior art process, involves the formation of (Z)-isomer, which causes low yield and affects the purity of the final product.

Therefore, there is a need to develop a process which provides Entacapone, which is operationally simple at an industrial scale and provides high yield and purity of final product.

With an objective of providing an improved process for the preparation of Entacapone of formula (I), the present inventors has directed the research work towards developing a process for preparing of Entacapone of formula (I) which devoid the drawback of the prior art.

Surprisingly, when the present inventors carried out the condensation of 3,4-dihydroxy-5-nitrobenzaldehyde of formula (II) and N,N-diethylcyanoacetamide of formula (III) using two component solvent system in the presence of catalyst to obtain Entacapone, the compound obtained by this process having high yield and good isomeric purity.

Moreover, this makes the process for the preparation of Entacapone operationally simple and easily applicable at an industrial scale.

### OBJECT OF THE INVENTION

Therefore, it is an object of the invention is to provide an improved process for the preparation of preparation of Entacapone of formula (I).

Another object of the invention is to provide an improved process for the preparation of Entacapone of formula (I) which is operationally simple, cost-effective, easy to handle and feasible at commercial scale.

Yet another object of the present invention is to provide an improved process for the preparation of Entacapone of formula (I).

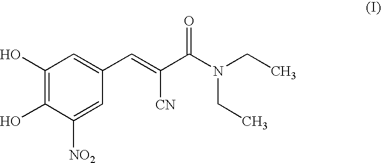

comprising a step of,
condensation of 3,4-dihydroxy-5-nitrobenzaldehyde of formula (II)

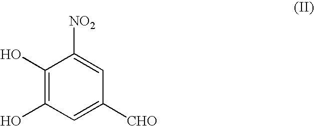

with N,N-diethylcyanoacetamide of formula (III)

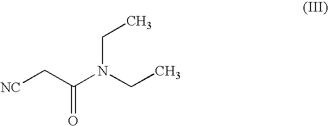

in the presence of two component solvent system, a catalyst and optionally a phase transfer catalyst to give Entacapone of formula (I).

The two component solvent system is selected from the group of solvent such as toluene and cyclohexane; toluene and acetonitrile; toluene and ethylacetate.

In another embodiment of the present invention, the two component solvent system may be comprised toluene and an ester.

The ester is defined hereinabove includes ethyl acetate, methyl acetate, butyl acetate, propyl acetate or the like and mixture thereof.

### DETAILED DESCRIPTION OF THE INVENTION

Accordingly, the present invention relates to an improved process for the preparation of Entacapone of formula (I)

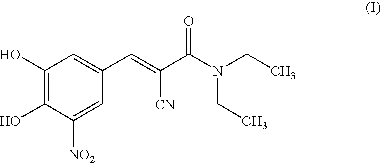

comprising a step of,
condensation of 3,4-dihydroxy-5-nitrobenzaldehyde of formula (II)

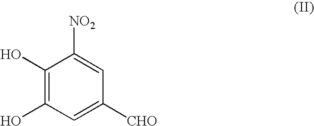

with N,N-diethylcyanoacetamide of formula (III)

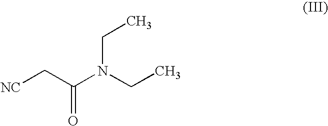

in the presence of two component solvent system, a catalyst and optionally a phase transfer, catalyst to give Entacapone of formula (I).

The two component solvent system is selected from the group of solvents such as toluene and cyclohexane; toluene and acetonitrile; toluene and ethylacetate.

The example of the suitable catalyst as mentioned hereinabove includes but not limited to inorganic base and organic base thereof.

The examples of the base mentioned hereinabove include but not limited to piperidine, pyridine, N-methylmorpholine, morpholine, piperazine and the like or mixture thereof.

The examples of the inorganic and organic salt of base mentioned hereinabove include but not limited sodium acetate, potassium t-butoxide, cesium t-butoxide, peperidinium acetate, pyridine acetate, piperidiniumpropionate and pyridinium para toluene sulfonate and the like or mixture thereof.

The examples of the phase transfer catalyst mentioned hereinabove in step (a) include but not limited to tetrabutylammonium bromide (TBAB), tetrabutylammonium hydroxide, TEBA, tricaprylylmethylammonium chloride, dodecyl sulfate sodium salt, tetrabutylammonium hydrogensulfate, hexadecyl tributyl phosphonium bromide, or hexadecyl trimethyl ammonium bromide.

When condensation process is carried out in the presence of two component solvent system, it provides final product of good yield as well as of high isomeric purity. The comparison of two component solvent system and single solvent system is shown below in Table-1.

TABLE 1

| S. No. | Solvent | Yield |
|---|---|---|
| 1 | Toluene | 57% |
| 2 | Ethanol | 73% |
| 3 | Toluene: Cyclohexane | 83% |

Therefore it is cleared from the observation that the two component solvent system provides a high yield and good isomeric purity of the final product.

The present invention provides process of preparation of Entacapone which is simple, environment friendly, economical and leads to an enhanced isomeric purity.

The process of the present invention has following advantages:
It provides a process which is economical, operational on and industrially applicable.
The process does not involve the use of corrosive material.
The process is simple and easy to handle and does not require special handling care or critical temperature conditions.
It eliminates the use of HBr which is harmful for health.
It reduces the period of time in reaction.
It does not require any specifically designed reactor.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims.

The process of the present invention is described by the following examples, which are illustrative only and should not be construed so as to limit the scope of the invention in any manner.

### Example 1

### Preparation of 3,4-dihydroxy 5-nitrobenzaldehyde

A mixture of anhydrous aluminum chloride (40.5 gm) under nitrogen atmosphere, pyridine (130 ml) and 5-nitro vanillin (50 gm) was charged at 5-10° C. and followed by heating at 50-55° C. After completion of reaction water (500 ml) and concentrated hydrochloric acid (150 ml) was added at 5-10° C. The reaction temperature was raised to 25-30° C. followed by addition of ethylacetate (500 ml). The layers were separated and organic layer was washed with saturated brine solution. Ethyl acetate was distilled out under reduced pressure and material was crystallized with cyclohexane (200 ml) and ethyl acetate (50 ml) to get 3,4-dihydroxy 5-nitrobenzaldehyde (41 gm).

### Example 2

### Preparation of Entacapone (Only Toluene)

3,4-dihydroxy 5-nitrobenzaldehyde (10 gm) and diethylcyanoacetamide (8.0 gm) was charged in toluene (100 ml) at rt followed by addition of piperidine (0.5 gm). The reaction temperature was raised to reflux (110-120° C.) and removed water azeotrophically from the reaction. After completion of reaction glacial acetic acid (20 ml) was added to reaction mixture followed by cooling at 25° C. to 30° C. The reaction mixture was filtered and washed with toluene and then with water. The residue was dried 50-55° C. to get Entacapone (9.45 gm).

HPLC Purity: E-isomer 99.02%, Z isomer content 0.12%.

### Example 3

### Preparation of Entacapone

3,4-dihydroxy 5-nitrobenzaldehyde (17 gm) and diethylcyanoacetamide (16.9 gm) was charged in a solution of toluene (85 ml) and cyclohexane (85 ml ml) at rt followed by addition of piperidine (0.78 gm). The reaction temperature was raised to reflux (88-94° C.) and removed water azeotrophically from the reaction. After completion of reaction glacial acetic acid (34 ml) was added to reaction mixture followed by cooling at 25° C. to 30° C. The reaction mixture was stirred and filtered. The residue was washed with toluene and water. The residue was dried under vacuum at 50-55° C. to get Entacapone (22.7 gm).

HPLC Purity: E-isomer 99.42% and Z isomer content 0.10%.

1. A process for the preparation of Entacapone of formula (I) 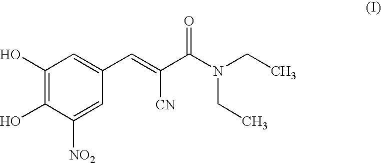
comprising a step of,
condensation of 3,4-dihydroxy-5-nitrobenzaldehyde of formula (II) 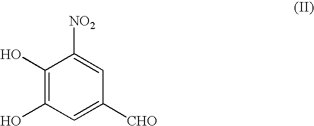
with N,N-diethylcyanoacetamide of formula (III) 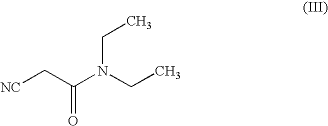
in the presence of two component solvent system, a catalyst and optionally a phase transfer catalyst to give Entacapone of formula (I).

2. A process according to claim 1, wherein the said catalyst is selected from the group comprising of organic base.

3. A process according to claim 2, wherein the said base is selected from the group comprising of piperidine, pyridine, N-methylmorpholine, morpholine, piperazine and the like or mixture thereof.

4. A process according to claim 2, wherein the said organic or inorganic salt of base is selected from the group comprising of sodium acetate, potassium t-butoxide, cesium t-butoxide, peperidinium acetate, pyridine acetate, piperidiniumpropionate and pyridinium para toluene sulfonate or mixture thereof.

5. A process according to claim 1, wherein the said two component solvent system is selected from the group of solvents such as toluene and cyclohexane; toluene and acetonitrile; toluene and ethylacetate.

6. A process according to claim 1, wherein the said phase transfer catalyst is selected from the group comprising of tetrabutylammonium bromide (TBAB), tetrabutylammonium hydroxide, TEBA, tricaprylylmethylammonium chloride, dodecyl sulfate sodium salt, tetrabutylammonium hydrogensulfate, hexadecyl tributyl phosphonium bromide, or hexadecyl trimethyl ammonium bromide.
